(19) United States
(12) Patent Application Publication
(10) Pub. No.: US 2018/0035870 A1
(43) Pub. Date: Feb. 8, 2018

(54) ENDOSCOPE

(71) Applicant: OLYMPUS CORPORATION, Tokyo (JP)

(72) Inventors: Suguru OKANIWA, Tokyo (JP); Seisuke TAKASE, Tokyo (JP); Hidehiro JOKO, Tokyo (JP); Isamu NAKAJIMA, Sagamihara-shi (JP)

(73) Assignee: OLYMPUS CORPORATION, Tokyo (JP)

(21) Appl. No.: 15/785,470

(22) Filed: Oct. 17, 2017

Related U.S. Application Data
(63) Continuation of application No. PCT/JP2016/083817, filed on Nov. 15, 2016.

(30) Foreign Application Priority Data
Nov. 17, 2015 (JP) 2015-224987

(51) Int. Cl. A61B 1/005; A61B 1/06; A61B 1/04

(52) U.S. Cl. CPC A61B 1/0057; A61B 1/04; A61B 1/06

(57) ABSTRACT

An endoscope including: a tube having flexibility and extending from a hand side to a distal end side along a longitudinal direction, the tube being provided to an insertion portion configured to be inserted into an object; and a strand formed by twining a plurality of wires each other and arranged so as to extend in the tube, the strand having a distal end portion fixed to a distal end side of the tube, wherein the strand is pulled from the hand side of the tube and a twining direction of the plurality of wires of the strand is reversed in a middle of extension in the tube.

### CROSS REFERENCE TO RELATED APPLICATION

This application is a continuation application of PCT/JP2016/083817 filed on Nov. 15, 2016 and claims benefit of Japanese Application No. 2015-224987 filed in Japan on Nov. 17, 2015, the entire contents of which are incorporated herein by this reference.

### BACKGROUND OF THE INVENTION

### 1. Field of the Invention

The present invention relates to an endoscope including a rigidity changing mechanism in an insertion portion.

### 2. Description of the Related Art

Endoscopes have been used in medical fields and industrial fields, for example, and such endoscopes include an image pickup unit for picking up an optical image in a distal end portion of an insertion portion configured to be insertable from an outside to an inside of a living body or structure in order to observe inside the living body or the structure where observation is difficult to perform.

The endoscope disclosed in Japanese Patent Application Laid-Open Publication No. 10-276965 has a rigidity changing mechanism that changes rigidity in a bending direction of a part of an insertion portion. The rigidity changing mechanism includes a coil pipe inserted in the insertion portion, a wire inserted in the coil pipe, and a pulling mechanism that applies a compression force to the coil pipe by pulling the wire. The rigidity in the bending direction of the coil pipe is changed depending on the compression force applied thereto. Therefore, the rigidity of the part of the insertion portion in which the coil pipe is inserted is changed depending on the compression force applied to the coil pipe.

### SUMMARY OF THE INVENTION

An endoscope according to one aspect of the present invention includes: a tube having flexibility and extending from a hand side to a distal end side along a longitudinal direction, the tube being provided to an insertion portion configured to be inserted into an object; and a strand formed by twining a plurality of wires each other and arranged so as to extend in the tube, the strand having a distal end portion fixed to a distal end side of the tube, wherein the strand is pulled from the hand side of the tube and a twining direction of the plurality of wires of the strand is reversed in a middle of extension in the tube.

In addition, an endoscope according to another aspect of the present invention includes: a tube having flexibility and extending from a hand side to a distal end side along a longitudinal direction, the tube being provided to an insertion portion configured to be inserted into an object; a coil spring arranged in the tube and formed by winding a spring wire around a predetermined axis parallel to the longitudinal direction; and a strand formed by twining a plurality of wires each other, inserted in the coil spring, and including a distal end portion fixed to the coil spring, wherein the plurality of wires constituting the strand are twined around a predetermined axis in a direction opposite to a winding direction of the spring wire of the coil spring.

### BRIEF DESCRIPTION OF THE DRAWINGS

FIG. 1 illustrates a configuration of an endoscope according to a first embodiment of the present invention.

FIG. 2 illustrates configurations of a flexible tube and a rigidity changing mechanism according to the first embodiment.

FIG. 3 illustrates winding directions of a coil spring, a first strand, and a second strand according to the first embodiment.

FIG. 4 illustrates another example of winding directions of the coil spring, the first strand, and the second strand according to the first embodiment.

FIG. 5 describes configurations of a flexible tube and a rigidity changing mechanism according to a second embodiment.

### DETAILED DESCRIPTION OF THE PREFERRED EMBODIMENT(S)

Hereinafter, preferable embodiments of the present invention will be described with reference to drawings. Note that, in the drawings to be used in the description below, a different scale size is used for each of constituent elements in order to allow the each of the constituent elements to be illustrated in a recognizable size, and the present invention is not limited to the number, shapes, ratio of the sizes of the constituent elements, and a relative positional relationship among the constituent elements shown in these drawings.

### First Embodiment

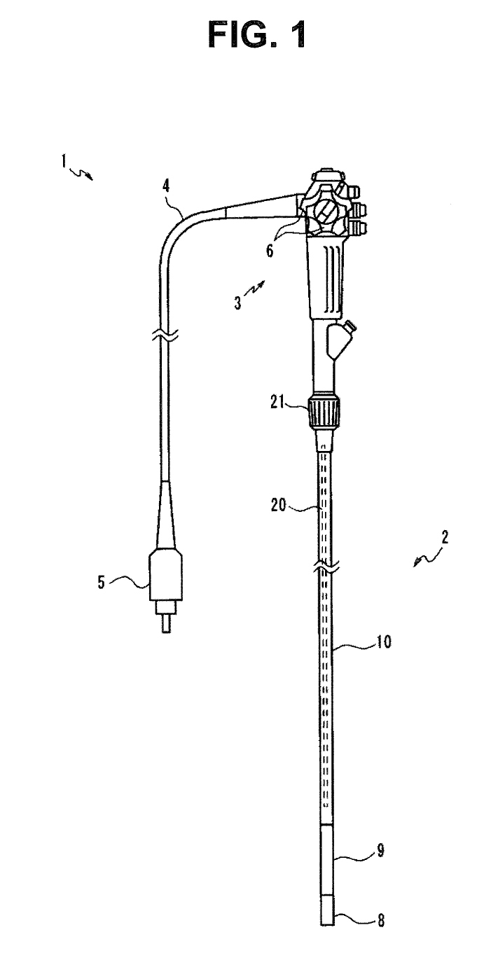

An endoscope 1 according to the present embodiment shown in FIG. 1 includes an elongated insertion portion 2 which is introduceable into a subject such as a human body, and the insertion portion 2 has a configuration for observing the inside of the subject. Note that the subject into which the insertion portion 2 of the endoscope 1 is introduced is not limited to a human body but may be another living body, or an artificial material such as a machine or construction.

The endoscope 1 according to the present embodiment mainly includes the insertion portion 2 formed in an elongated shape and configured to be introduced into an inside of a subject, an operation portion 3 positioned at a proximal end of the insertion portion 2, and a universal cord 4 extended from the operation portion 3.

The insertion portion 2 includes in a linked manner: a distal end portion 8 disposed at a distal end; a bendable bending portion 9 disposed on a proximal end side of the distal end portion 8; and a flexible tube 10 having flexibility and connecting the proximal end side of the bending portion 9 and the distal end side of the operation portion 3.

The distal end portion 8 includes a configuration for observing an inside of a subject. For example, the distal end portion 8 includes an image pickup unit including an objective lens and an image pickup device and configured to optically observe the inside of the subject. In addition, the distal end portion 8 is provided with an illumination light emission portion that emits light for illuminating the object of the image pickup unit, though not shown. Note that the distal end portion 8 may be provided with an ultrasound transducer for acoustically observing the inside of the subject using ultrasound.

The operation portion 3 disposed at the proximal end of the insertion portion 2 includes an angle operation knob 6 for operating the bending of the bending portion 9. At the proximal end portion of the universal cord 4, an endoscope connector 5 which is configured to be connectable to an external apparatus, not shown, is provided. The external apparatus to which the endoscope connector 5 is connected includes a camera control unit for controlling the image pickup unit provided at the distal end portion 8, and the like.

In addition, the operation portion 3 is provided with a rigidity changing knob 21 for operating a rigidity changing mechanism 20 disposed in the flexible tube 10. The rigidity changing mechanism 20 is inserted into the flexible tube 10 along the longitudinal direction of the flexible tube 10, and configured such that the rigidity against flexion changes according to the operation input through the rigidity changing knob 21. That is, the rigidity changing mechanism 20 changes the rigidity against the flexion of the flexible tube 10.

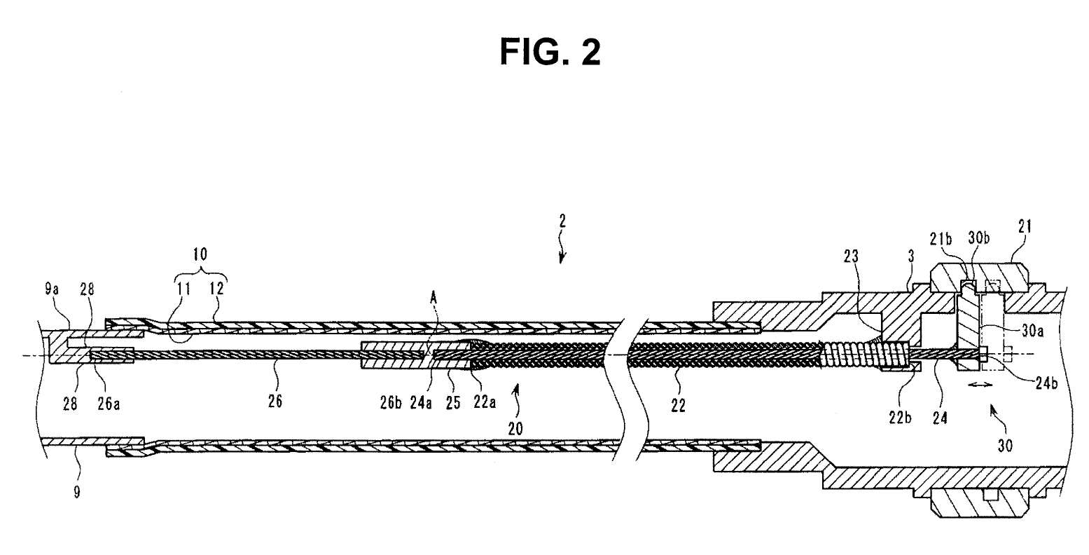

Next, description will be made on the flexible tube 10 and the rigidity changing mechanism 20. As shown in FIG. 2, the flexible tube 10 includes a mesh tube 11 and an outer cover 12.

The mesh tube 11 is formed by braiding a thin wire made of metal such as stainless alloy in a tubular shape. The outer cover 12 is a coat made of synthetic resin that covers the outer circumference of the mesh tube 11. The mesh tube 11 is covered with the outer cover 12, to thereby keep the airtightness in the flexible tube 10.

Note that a flex tube which is a core material that prevents crushing of the flexible tube 10 is provided inside the mesh tube 11, though not shown. The flex tube is formed by helically winding an elongated thin metal plate around an axis that is along the longitudinal direction of the flexible tube 10. The width of the thin plate that configures the flex tube is narrower than a width of a pitch at which the thin plate is wound. The flex tube deforms according to the flexion of the flexible tube 10.

Inside the flexible tube 10 configured as described above, internal components such as an electric cable that electrically connects the image pickup unit and the endoscope connector and a conduit through which a fluid or a treatment instrument passes are inserted, in addition to the above-described rigidity changing mechanism 20. Since the internal components other than the rigidity changing mechanism 20 are known techniques, the descriptions thereof will be omitted.

The rigidity changing mechanism 20 includes a coil spring 22, a first strand 24, and a second strand 26. With regard to the members that constitute the rigidity changing mechanism 20, the direction toward the distal end portion 8 of the insertion portion 2 is referred to as a distal end direction, and the direction toward the operation portion 3 is referred to as a proximal end direction.

The coil spring 22 is formed in a cylindrical shape by helically winding a spring wire made of metal such as stainless alloy around a predetermined axis A that is parallel to the longitudinal direction of the insertion portion 2.

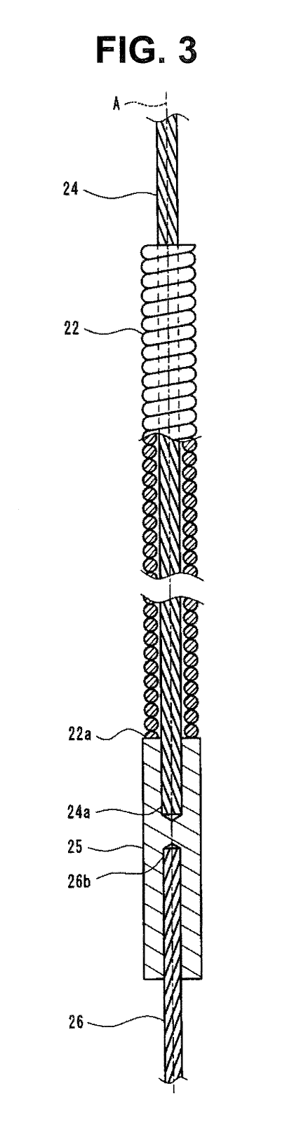

The coil spring 22 in the present embodiment is formed by winding a spring wire into a right-handed helix, as one example as shown in FIG. 3. The right-handed helix is a helix having a winding direction in which when the coil spring 22 is placed such that the axis A extends in the vertical direction as shown in FIG. 3, the part of the spring wire, which is shown on the front side of the coil spring 22, rises diagonally up to the right. Such a winding direction is also referred to as a Z-winding.

A proximal end 22b of the coil spring 22 is held by a coil spring fixing portion 23 fixed to the operation portion 3. The coil spring 22 includes inside thereof a space having a predetermined internal diameter with the predetermined axis A as a center. The first strand 24 to be described later is inserted in the coil spring 22.

The first strand 24 is inserted in the coil spring 22 and formed by twining a plurality of wires each other around the predetermined axis A in a direction opposite to the winding direction of the coil spring 22. The first strand 24 is formed by twining the plurality of wires made of metal such as stainless alloy each other, for example.

In the present embodiment, the coil spring 22 is wound into the right-handed helix as described above. Therefore, the first strand 24 is formed by twining the plurality of wires into a left-handed helix. The left-handed helix is a helix having a winding direction in which when the first strand 24 is placed such that the axis A extends in the vertical direction as shown in FIG. 3, the part of the plurality of wires, which is shown on the front side of the first strand 24, rises diagonally up to the left. Such winding and twining are also referred to respectively as S-winding, S-twining, and the like.

A distal end 24a of the first strand 24 inserted in the coil spring 22 is integrated with a distal end 22a of the coil spring 22 such that a compression force in a direction in which the coil spring 22 is compressed along the axis A is applied to the coil spring 22, when the first strand 24 is pulled in the proximal end direction.

Specifically, the distal end 24a of the first strand 24 protrudes further in the distal end direction than the distal end 22a of the coil spring 22. A connection member 25 having an outer diameter larger than the inner diameter of the coil spring 22 is secured to the distal end 24a of the first strand 24. That is, the relative movement of the distal end 24a of the first strand 24 in the proximal end direction with respect to the distal end 22a of the coil spring 22 is restricted by the connection member 25.

In addition, the connection member 25 is secured to the distal end 22a of the coil spring 22 by adhesive, soldering, brazing, or the like. That is, the distal end 24a of the first strand 24 is fixed to the distal end 22a of the coil spring 22. Note that the distal end 24a of the first strand 24 may be directly secured to the distal end 22a of the coil spring 22 by adhesive, soldering, brazing, or the like without using the connection member 25.

As described above, the proximal end 22b of the coil spring 22 is fixed to the operation portion 3 by the coil spring fixing portion 23. Therefore, when the first strand 24 is pulled in the proximal end direction, a tensile force applied to the first strand 24 is transferred to the distal end 22a of the coil spring 22, and the force for compressing the coil spring 22 in the direction of the axis A is applied to the coil spring 22. The compression force is applied to the coil spring 22, to thereby increase a resistance force against the bending deformation, which is generated by the coil spring 22. The greater the compression force to be applied to the coil spring 22, the greater the resistance force against the bending deformation generated by the coil spring 22.

The proximal end 24b of the first strand 24 is connected to a pulling mechanism 30 that pulls the first strand 24 in the proximal end direction and applies the tensile force to the first strand 24.

Since the pulling mechanism 30 is known, detailed description thereof will be omitted. In the present embodiment, as one example, the pulling mechanism 30 includes a rigidity changing knob 21 that rotates with respect to the operation portion 3, and a wire holding portion 30a that holds the proximal end 24b of the first strand 24 and advances and retracts in the direction along the axis A according to the rotation of the rigidity changing knob 21.

A cam groove 21b is carved on the inner circumferential surface of the rigidity changing knob 21. The wire holding portion 30a is disposed so as to be able to advance and retract in the direction along the axis A in the operation portion 3. In addition, the wire holding portion 30a is provided with a cam pin 30b that is slidably engaged with the cam groove 21b. Engagement between the cam groove 21b and the cam pin 30b allows the wire holding portion 30a to advance and retract in the direction along the axis A according to the rotation of the rigidity changing knob 21. The pulling mechanism 30 thus configured according to the present embodiment is capable of changing the tensile force to be applied to the first strand 24 in accordance with the rotation operation of the rigidity changing knob 21 by the user.

The second strand 26 is formed by winding a plurality of wires around the predetermined axis A in the direction opposite to the twining direction of the first strand 24. That is, the second strand 26 is formed by twining the plurality of wires around the predetermined axis A in the direction same as the winding direction of the coil spring 22. The second strand 26 is formed by twining the plurality of wires made of metal such as stainless alloy each other, for example.

In the present embodiment, as described above, the first strand 24 is twined in a left-handed fashion. Therefore, the second strand 26 is formed by twining the plurality of wires into the right-handed helix. The right-handed twining is also referred to as Z-winding or Z-twining.

The second strand 26 is fixed to the constituent element of the insertion portion 2, with the rotation of the distal end 26a being restricted, and the proximal end 26b is fixed to the distal end 24a of the first strand 24. That is, the second strand 26 is arranged on the distal end side with respect to the first strand 24.

Specifically, the distal end 26a of the second strand 26 is fixed to a strand fixing portion 28 of a frame member 9a arranged at the proximal end of the bending portion 9 of the insertion portion 2, with the rotation around the axis A being restricted. The distal end 26a of the second strand 26 is secured to the strand fixing portion 28 by adhesive, soldering, brazing, or the like, for example.

In addition, the proximal end 26b of the second strand 26 is secured to the connection member 25 by adhesive, soldering, brazing, or the like, for example. The distal end 24a of the first strand 24 and the proximal end 26b of the second strand 26 are secured to the connection member 25 such that the central axes thereof are located on the axis A.

Note that the proximal end 26b of the second strand 26 may be directly secured to the distal end 24a of the first strand 24 by adhesive, soldering, brazing, or the like, for example, without using the connection member 25.

The distal end 26a of the second strand 26 is secured to the frame member 9a of the bending portion 9, which allows the position of the distal end 24a of the first strand 24 in the flexible tube 10 to be held within a predetermined range. That is, the range in which the distal end 22a of the coil spring 22 can be moved in the flexible tube 10 is determined by the second strand 26. Therefore, the coil spring 22 is held so as to be movable in the flexible tube 10 only in the range determined by the second strand 26 in the case where the first strand 24 is pulled in the proximal end direction by the pulling mechanism 30 and flexion of the flexible tube 10 is repeated.

The resistance force of the coil spring 22 against the bending deformation changes according to the tensile force applied to the first strand 24 by the pulling mechanism 30. Therefore, the rigidity against the flexion of the flexible tube 10 in the range in which the coil spring 22 is arranged changes according to the resistance force against the bending deformation of the coil spring 22. According to the configuration as described above, the rigidity changing mechanism 20 changes the rigidity of at least a part of the insertion portion 2.

As described above, the rigidity changing mechanism 20 included in the endoscope 1 according to the present embodiment is provided with the coil spring 22 formed by winding the spring wire around the predetermined axis A, the first strand 24 twined around the axis A in the direction opposite to the winding direction of the coil spring 22, the second strand 26 twined in the direction opposite to the twining direction of the first strand 24, and the pulling mechanism 30 that pulls the first strand 24.

In addition, the first strand 24 is inserted in the coil spring 22, the distal end 24a of the first strand 24 is fixed to the distal end 22a of the coil spring 22, and the proximal end 24b of the first strand 24 is connected to the pulling mechanism 30. In addition, the second strand 26 is engaged with the constituent element of the insertion portion 2, with the rotation of the distal end 26a being restricted, and the proximal end 26b is fixed to a portion at which the first strand 24 and the coil spring 22 are fixed to each other. The pulling mechanism 30 applies a compression force to the coil spring 22 by pulling the first strand 24 in the proximal end direction.

In the rigidity changing mechanism 20 thus configured according to the present embodiment, when the tensile force is applied to the first strand 24 by the pulling mechanism 30, the first strand 24 generates a rotational force F1 around the axis A by being untwined. The deformation due to the untwining of the first strand 24 can be reworded as the deformation due to extension of the first strand 24.

On the other hand, when the tensile force is applied to the first strand 24 by the pulling mechanism 30, the compression force is applied to the coil spring 22, and the coil spring 22 generates a rotational force F2 around the axis A by being deformed so that the total number of windings of the coil spring 22 is decreased. The deformation due to the decrease in the total number of windings of the coil spring 22 can be reworded as the deformation due to the contraction of the coil spring 22.

The winding direction of the coil spring 22 and the twining direction of the first strand 24 are in the relationship opposite to each other. When the tensile force is applied to the first strand 24 by the pulling mechanism 30, the rotational force F1 generated by the first strand 24 and the rotational force F2 generated by the coil spring 22 are in a relationship in which the rotational forces act in directions so as to cancel each other. Therefore, the rigidity changing mechanism 20 according to the present embodiment is capable of suppressing or preventing the amount of deformation that occurs when the coil spring 22 contracts and the amount of deformation that occurs when the first strand 24 extends, when a tensile force is applied to the first strand 24, thereby being capable of suppressing or preventing the contraction of the free length of the coil spring 22 and the extension of the free length of the first strand 24 which are caused by repeated use.

In addition, the rigidity changing mechanism 20 according to the present embodiment includes the second strand 26 twined in the direction opposite to the twining direction of the first strand 24. When the first strand 24 is pulled by the pulling mechanism 30 in the proximal end direction, a tensile force is applied also to the second strand 26. Since the second strand 26 is in the state where the rotation of the distal end 26a is restricted, when the proximal end 26b is pulled in the proximal end direction, the second strand 26 generates a rotational force F3 around the axis A by being untwined.

The twining direction of the first strand 24 and the twining direction of the second strand 26 are in the relationship opposite to each other. Therefore, when the tensile force is applied to the first strand 24 by the pulling mechanism 30, the rotational force F1 generated by the first strand 24 and the rotational force F3 generated by the second strand 26 are in the relationship in which the rotational forces act in directions so as to cancel each other. The rigidity changing mechanism 20 according to the present embodiment is capable of suppressing or preventing the change in the twining of the first strand 24 and second strand 26 when the tensile force is applied to the first strand 24. As a result, extension of the free lengths of the first strand 24 and the second strand 26, which is caused by the repeated use, can be suppressed or prevented.

In addition, the rigidity changing mechanism 20 according to the present embodiment is capable of suppressing or preventing the change in the twining of the first strand 24 and second strand 26 when the tensile force is applied to the first strand 24, thereby capable of preventing kink caused by the second strand 26 being untwined too much or twined too tightly.

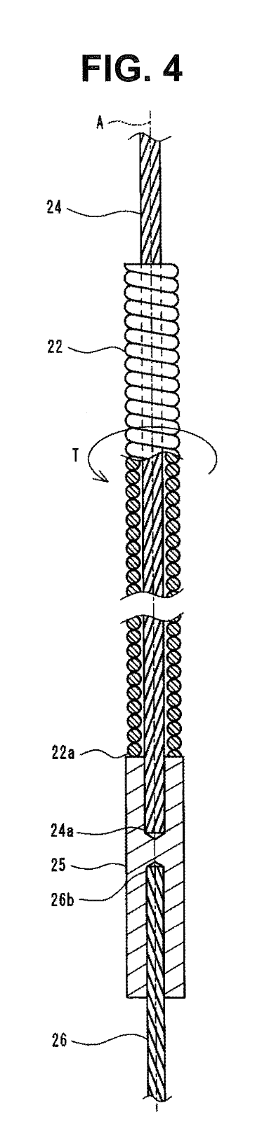

Note that, in the present embodiment described above, the coil spring 22 and the second strand 26 are right-handed (Z-winding), and the first strand 24 is left-handed (S-winding). However, the winding directions may be reversed. That is, as shown in FIG. 4, even if the coil spring 22 and the second strand 26 are left-handed (S-winding) and the first strand 24 is right-handed (Z-winding), the same working and effects as those in the above-described embodiment can be obtained.

### Second Embodiment

Hereinafter, the second embodiment of the present invention will be described. Hereinafter, only the points different from the first embodiment will be described. The same constituent elements as those in the first embodiment are attached with the same reference numerals and descriptions thereof will be omitted.

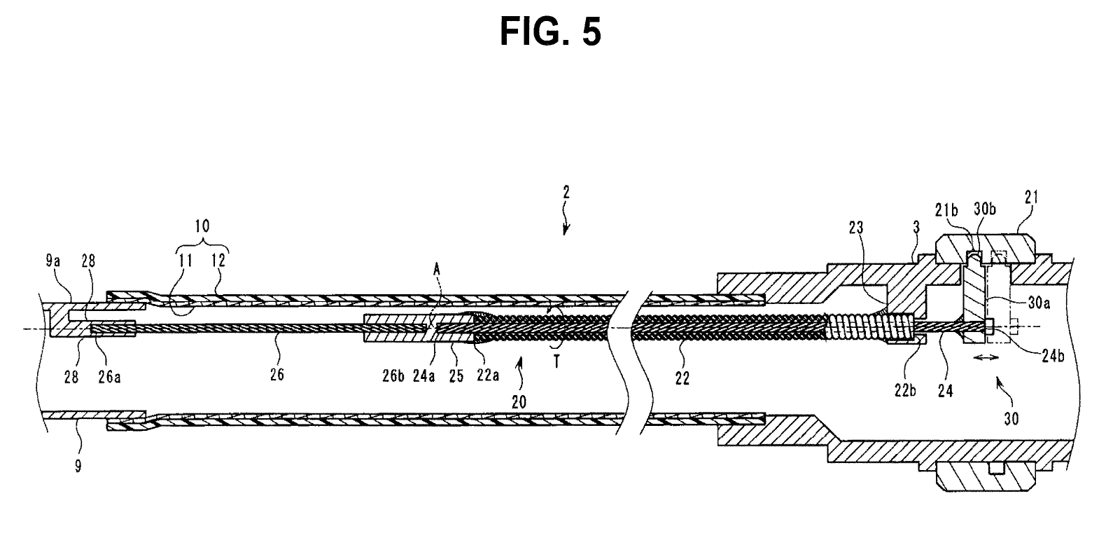

The endoscope 1 according to the present embodiment shown in FIG. 5 is different from the one in the first embodiment in that the coil spring 22 of the rigidity changing mechanism 20 is applied with a twisting force T around the axis A in a direction same as the winding direction of the coil spring 22.

In the present embodiment shown in the drawings, as one example, the coil spring 22 is formed by winding a spring wire right-handedly (Z winding). The coil spring 22 according to the present embodiment is configured such that the distal end 22a is fixed to the distal end 24a of the first strand 24 and the proximal end 22b is fixed to the coil spring fixing portion 23, in the state where the coil spring 22 is twisted by a predetermined angle from the natural state in the direction same as the winding direction.

Twisting the coil spring 22 in the direction same as the winding direction means that twisting the coil spring 22 around the axis A in the direction in which the total number of windings of the coil spring 22 increases. Arranging the coil spring 22 in the state twisted in the direction same as the winding direction thereof causes the resistance force against the bending deformation, which is generated by the coil spring 22, to be greater than in the case where the coil spring 22 is in the natural state.

Therefore, in the present embodiment, the initial rigidity of the coil spring 22 in the state where the first strand 24 is not pulled by the pulling mechanism 30 can be adjusted by changing the magnitude of the twisting force T to be applied to the coil spring 22. Adjusting the initial rigidity of the coil spring 22 enables the variation of the rigidities of the flexible tubes 10 which occurs in a plurality of endoscopes 1 to be suppressed.

In addition, in the present embodiment, similarly as in the first embodiment, the winding direction of the coil spring 22 and the twining direction of the second strand 26 are the same, and the twining direction of the first strand 24 is opposite to the winding direction of the coil spring 22. Therefore, also in the endoscope 1 according to the present embodiment, similarly as in the first embodiment, when the tensile force is applied to the first strand 24, the amount of deformation that occurs when the coil spring 22 contracts and the amount of deformation that occurs when the first strand 24 extends can be suppressed or prevented, and the contraction of the free length of the coil spring 22 and the extension of the free length of the first strand 24 can be suppressed or prevented. In addition, the endoscope according to the present embodiment is capable of suppressing or preventing the change in the twining of the first strand 24 and second strand 26 when the tensile force is applied to the first strand 24, thereby capable of preventing kink caused by the second strand 26 being untwined too much or twined too tightly.

The present invention is not limited to the above-described embodiments, and appropriate changes are possible without departing from the gist or thought of the invention that can be read from claims and throughout the specification, and endoscopes with such changes are also included in the technical range of the present invention.

What is claimed is:

1. An endoscope comprising:
a tube having flexibility and extending from a hand side to a distal end side along a longitudinal direction, the tube being provided to an insertion portion configured to be inserted into an object; and
a strand formed by twining a plurality of wires each other and arranged so as to extend in the tube, the strand having a distal end portion fixed to a distal end side of the tube, wherein the strand is pulled from the hand side of the tube and a twining direction of the plurality of wires of the strand is reversed in a middle of extension in the tube.

2. The endoscope according to claim 1, further comprising:
a cylindrical member including a distal end portion fixed to a part at which the twining direction of the plurality of wires of the strand is reversed, and a proximal end portion fixed to a proximal end side of the tube; and
a pulling mechanism arranged on the proximal end side of the tube, the pulling mechanism pulling the strand in a proximal end direction to apply a compression force to the cylindrical member.

3. The endoscope according to claim 2, wherein the strand is configured by connecting ends of two strands including a plurality of wires, twining directions of which are different from each other.

4. The endoscope according to claim 2, wherein the cylindrical member changes an amount of force required for bending the tube by a compressed state of the cylindrical member being changed.

5. The endoscope according to claim 1, wherein the cylindrical member is configured of a coil spring formed by winding a spring wire around a predetermined axis parallel to the longitudinal direction.

6. The endoscope according to claim 5, wherein the coil spring is arranged in the tube in a state being applied with a twisting force in a direction same as the winding direction.

7. An endoscope comprising:
a tube having flexibility and extending from a hand side to a distal end side along a longitudinal direction, the tube being provided to an insertion portion configured to be inserted into an object;
a coil spring arranged in the tube and formed by winding a spring wire around a predetermined axis parallel to the longitudinal direction; and
a strand formed by twining a plurality of wires each other, inserted in the coil spring, and including a distal end portion fixed to the coil spring, wherein the plurality of wires constituting the strand are twined around a predetermined axis in a direction opposite to a winding direction of the spring wire of the coil spring.

8. The endoscope according to claim 7, further comprising
another strand,
wherein the other strand is formed by twining a plurality of wires each other, and including a proximal end fixed to a portion at which the distal end portion of the strand and the coil spring are fixed to each other, and a distal end fixed to a constituent member of the tube, and the plurality of wires constituting the other strand are twined in a direction same as the winding direction of the spring wire of the coil spring.

9. The endoscope according to claim 8, wherein the coil spring is arranged in the tube in the state being applied with a twisting force in a direction same as the winding direction of the spring wire.

10. The endoscope according to claim 8, further comprising,
a pulling mechanism that pulls the strand in a proximal end direction to apply a compression force to the coil spring.

11. The endoscope according to claim 10, wherein the coil spring changes an amount of force required for bending the tube by a compressed state of the coil spring being changed.

12. The endoscope according to claim 7, wherein the coil spring is arranged in the tube in the state being applied with a twisting force in a direction same as the winding direction of the spring wire.
